(19) United States
(12) Patent Application Publication
(10) Pub. No.: US 2026/0335213 A1
(43) Pub. Date: Oct. 29, 2026

(54) Solid Anhdrous Composition Comprising Ethylcellulose, an Alkyl Benzoate and a Silicone Oil and Process Employing Same

(71) Applicant: L'OREAL, Paris (FR)

(72) Inventors: Emilie HENIN, Chevilly Larue (FR); Florence LAHOUSSE, Chevilly Larue (FR); Sylvie GUILLARD, Chevilly Larue (FR)

(73) Assignee: L'OREAL, Paris (FR)

(21) Appl. No.: 19/099,149

(22) PCT Filed: Jul. 28, 2023
(86) PCT No.: PCT/EP2023/070954

§ 371 Date: Jan. 28, 2025

(30) Foreign Application Priority Data
Jul. 29, 2022 (FR) 2207928

(51) Int. Cl. A61K 8/73; A61K 8/31; A61K 8/37; A61K 8/891; A61Q 1/04

(52) U.S. Cl. CPC A61K 8/731; A61K 8/31; A61K 8/37; A61K 8/891; A61Q 1/04; A61K 2800/43; A61K 2800/48

(57) ABSTRACT

The invention relates to a solid composition for making up human keratin materials, in particular the skin and the lips, comprising: ethylcellulose; at least a first oil, which is chosen from esters of benzoic acid and of a C₈-C₂₀ monoalcohol, at least a second oil, which is a polar non-volatile hydrocarbon-based oil chosen from C₁₀-C₂₆ alcohols and optionally from esters other than the first oil(s), at least a third oil, which is chosen from non-volatile phenylated silicones comprising at least one dimethicone fragment, optionally at least a fourth oil, which is a non-volatile non-phenylated silicone oil or a non-volatile phenylated silicone oil with no dimethicone fragment, the contents of oils being such that the weight ratio of the first oil(s), second oil(s) and where appropriate fourth oil(s)/third oil(s) is less than or equal to 1. The invention also relates to a process for making up the skin or the lips, in which the above-mentioned composition is applied.

A subject of the present invention is a solid composition for making up keratin materials, in particular the skin and/or the lips, comprising ethylcellulose, at least one benzoate ester oil, at least one liquid fatty alcohol, and at least one phenylated silicone oil comprising a dimethicone fragment, in specific contents. Another subject of the invention is a process for making up and/or caring for in particular the lips, consisting in employing such a composition.

The present invention is more particularly concerned with compositions for making up the skin and/or the lips for which it is desired to obtain a glossy finish.

Liquid or solid anhydrous compositions, dedicated in particular to making up human keratin materials, such as the skin and the lips, with a glossy result, are obtained from mixtures comprising relatively high contents of oils, in particular non-volatile oils, and also colorants, such as, for example, pearlescent agents and/or pigments.

In the case of fluid compositions, they have to exhibit a viscosity sufficient to guarantee stability of said compositions over time, in particular to keep the colorants in suspension and to prevent the composition from flowing out of the areas to be treated or to be made up during application. The viscosity of the compositions must also limit the migration of the latter into the wrinkles and fine lines of the outline of the lips after application. However, this viscosity must remain such that the compositions can be applied satisfactorily, with good slip, in order to obtain a homogeneous deposited layer which is sufficiently thin.

In the case of solid compositions, structuring agents, such as, for example, waxes, are often added to the abovementioned mixtures. These compounds have to sufficiently stiffen the compositions for them to be able to be moulded in the stick form, without harming their properties of use. This is because the compositions have to be capable of being destructured in order to make possible the ready application of an appropriate amount to the lips. Anhydrous makeup compositions comprising a first oil, for instance octyldodecanol, and at least one non-volatile silicone oil not compatible with the first oil, and ethylcellulose are known for example in document WO 2017/108584.

These compositions make it possible to obtain a glossy deposit which remains over time. However, it would be preferable to further improve the transfer resistance of the deposit obtained, without however degrading the properties of gloss or comfort of the resulting deposit, or increasing the tacky feeling or introducing a feeling of tautness or dryness of the lips once the composition has been applied.

These problems and others are solved by the present invention, a subject of which is thus a solid cosmetic composition for making up and/or caring for human keratin materials, in particular the lips, comprising:
at least ethylcellulose,
at least a first oil, which is chosen from monoesters of benzoic acid and of a monoalcohol, which is preferably saturated, of C₈-C₂₀, preferably C₈-C₁₈, and also mixtures thereof,
at least a second oil, which is a polar non-volatile hydrocarbon-based oil chosen from C₁₀-C₂₆ alcohols and optionally from esters other than the first oil(s),
at least a third oil, which is chosen from non-volatile phenylated silicones comprising at least one dimethicone fragment,
optionally at least a fourth oil, which is a non-phenylated silicone oil or phenylated silicone oil with no dimethicone fragment,
the contents of oils being such that the weight ratio of the first oil(s), second oil(s) and where appropriate fourth oil(s)/third oil(s) is less than or equal to 1.

Another subject of the invention is a process for making up human keratin materials, in particular the skin or the lips, in which the abovementioned composition is applied.

The composition according to the invention exhibits the advantage of being stable over time and being easy to apply, with improved glide. Moreover, the deposit obtained is precise and uniform, does not stick and does not migrate into the wrinkles and fine lines, in particular around the lips, or the eyes (crow's feet).

The resulting deposit is moreover fine, despite a high content of non-volatile oils. The deposit makes it possible to obtain a glossy makeup result, with an improved wear property. It is also comfortable, without leaving a feeling of dryness or tautness.

Finally, the transfer resistance of the deposit obtained by application of the composition according to the invention is improved.

In that which will follow, the expression “at least one” is equivalent to “one or more”.

The expressions “between . . . and . . . ” and “ranging from . . . to . . . ” should be understood as meaning limits included, unless otherwise specified.

The temperatures mentioned in the description are shown at atmospheric pressure (1.013×10⁵ Pa).

Since the composition according to the invention is a cosmetic composition, this means in particular that it comprises a physiologically acceptable medium, that is to say a medium that is particularly suitable for the application of a composition of the invention to human keratin materials, most particularly the skin and the lips.

The physiologically acceptable medium is generally adapted to the nature of the support onto which the composition is to be applied, and also to the form in which the composition is to be packaged.

The composition according to the invention preferably comprises less than 5% by weight, more particularly less than 2% by weight of water, or even less than 1% by weight of water, relative to the total weight of the composition. Advantageously, the composition according to the invention is anhydrous. The term “anhydrous” is understood in particular to mean that water is not deliberately added to the composition but may be present in the trace form in the various compounds used.

The composition according to the invention is in a solid form. The term “solid” is understood to mean in particular a composition that does not flow under its own weight at ambient temperature (25 °C) and atmospheric pressure. Preferably, the composition according to the invention is in the form of a wand. More particularly, the term “wand” is understood to mean a pharmaceutical form, the hardness of which can be measured according to the “cheese wire” method below:

### Protocol for Measuring the Hardness:

The composition in wand form is stored at 20 °C for 24 hours before measuring the hardness.

The measurement is performed at 20 °C and consists in transversely cutting a wand of product, which is preferably a circular cylinder, by means of a rigid tungsten wire 250 μm in diameter, by moving the wire relative to the stick at a speed of 100 mm/minute.

The hardness of the samples of compositions of the invention, expressed in Nm-1, is measured using a DFGS2 tensile testing device sold by Indelco-Chatillon.

The measurement is repeated three times and then averaged. The average of the three values read using the tensile testing machine mentioned above, noted Y, is given in grams. This average is converted into newtons and then divided by L which represents the longest distance through which the wire passes. In the case of a cylindrical wand, L is equal to the diameter (in metres).

The hardness is converted into Nm⁻¹ by the following equation: (Y×10⁻³×9.8)/L

According to this measurement method, the composition according to the invention, when it is in solid form, advantageously exhibits a hardness at 20 °C and at atmospheric pressure of between 20 and 150 Nm⁻¹ and preferably between 40 and 120 Nm⁻¹.

### Ethylcellulose

As indicated previously, the composition according to the invention comprises ethylcellulose.

Ethylcellulose is a cellulose alkyl ether comprising a chain formed from β-anhydroglucose units linked together via acetal bonds. Each anhydroglucose unit contains three replaceable hydroxyl groups, all or some of these hydroxyl groups being able to react according to the following reaction:

RONa+R′Cl ROR′+NaCl, where R represents a cellulose radical and R′ represents an ethyl radical.

Total substitution of the three hydroxyl groups would lead, for each anhydroglucose unit, to a degree of substitution of 3, in other words to a content of alkoxy groups of 54.88%.

The ethylcellulose polymers used in a cosmetic composition according to the invention are preferentially polymers with a degree of substitution with ethoxy groups ranging from 2.5 to 2.6 per anhydroglucose unit, in other words comprising a content of ethoxy groups ranging from 44% to 50%.

The ethylcellulose used in the composition according to the invention is more particularly found in powdered form.

It is sold, for example, under the trade names Ethocel Standard from Dow Chemicals, notably including Ethocel Standard 7 FP Premium and Ethocel Standard 100 FP Premium. Other commercially available products, such as those sold by Ashland, Inc. under the trade names Aqualon EC type-K, type-N and type-T, preferably type-N, such as N7, N100, are particularly suitable for performing the invention.

Advantageously, the ethylcellulose content ranges from 1% to 10% by weight and preferably from 2% to 6% by weight relative to the total weight of the composition.

The composition according to the invention comprises, in addition to the ethylcellulose, at least one particular benzoic acid ester (oil 1), at least a second oil which is a polar non-volatile hydrocarbon-based oil other than oil 1, among which is at least one fatty alcohol (oil 2), at least one non-volatile phenylated silicone oil with no dimethicone fragment (oil 3) and optionally at least one different non-volatile silicone oil (oil 4). The respective contents of the oils in the composition is such that the weight ratio of the first oil(s), second oil(s) and where appropriate fourth oil(s)/third oil(s) is between 0.5 and 1, more particularly between 0.7 and 1.

### Benzoic Acid Ester First Oils

The composition according to the invention comprises at least a first oil (therefore a compound which is liquid at ambient temperature and atmospheric pressure) which is chosen from esters of benzoic acid and of a monoalcohol, which is preferably saturated, of C₈-C₂₀, preferably C₈-C₁₈, and also mixtures thereof.

More particularly, the first oil is chosen from C₁₂-C₁₅ alkyl benzoate (C₁₂-15 Alkyl Benzoate-INCI name), C₁₆-C₁₇ alkyl benzoate (C₁₆-17 Alkyl Benzoate-INCI name), stearyl benzoate, isostearyl benzoate, ethylhexyl benzoate and octyldodecyl benzoate, and also mixtures thereof, and preferably C₁₂-C₁₅ alkyl benzoate.

Advantageously, the content of first oil(s) (C) is from 8% to 30% by weight, more particularly from 10% to 25% by weight, relative to the total weight of the composition.

### Polar Non-Volatile Hydrocarbon-Based Second Oils

As indicated above, the composition according to the invention comprises at least a second oil, which is a polar non-volatile hydrocarbon-based oil.

The term “oil” is understood to mean a lipophilic compound which is liquid at ambient temperature (25 °C) and atmospheric pressure (1.013×10⁵ Pa).

Advantageously, the oil is water-immiscible. The term “water-immiscible” is understood to mean that the mixing of the same amount of water and oil, after stirring, does not result in a stable solution comprising only a single phase, under normal temperature and pressure conditions. Observation is performed by eye or using a phase-contrast microscope, if necessary, on 100 g of mixture obtained after sufficient stirring with a Rayneri blender to produce a vortex within the mixture (as a guide, 200 to 1000 rpm), the resulting mixture being left to stand, in a closed flask, for 24 hours at ambient temperature before observation.

The term “hydrocarbon-based oil” is understood to mean an oil formed essentially from, or even constituted of, carbon and hydrogen atoms, and possibly one or more oxygen and nitrogen atom(s), and not containing other heteroatoms such as, for example, one or more silicon atoms. The hydrocarbon-based oil is thus different from a silicone oil. It may contain alcohol, ester, ether, carboxylic acid, amine and/or amide groups.

A polar oil for the purposes of the invention comprises, in addition to the carbon and hydrogen atoms, at least one oxygen or nitrogen atom and preferably at least one oxygen atom.

The term “non-volatile oil” is understood to mean an oil that has a vapour pressure of less than 2.66 Pa, preferably less than 0.13 Pa. By way of example, the vapour pressure may be measured via the static method or via the effusion method by isothermal thermogravimetry, depending on the vapour pressure (OECD standard 104).

The composition according to the invention comprises, as second oil, at least one C₁₀-C₂₆ alcohol and optionally at least one oil chosen from esters other than the first oil(s).

C₁₀-C₂₆ Alcohols

The alcohols are more particularly fatty alcohols. They comprise a linear or branched, saturated or unsaturated hydrocarbon-based radical comprising from 10 to 26 carbon atoms, preferably comprising from 10 to 24 carbon atoms and more preferentially from 12 to 22 carbon atoms. They are preferably monohydroxylated.

Preferably, said alcohol(s) is/are chosen from lauryl alcohol, isostearyl alcohol, oleyl alcohol, 2-butyloctanol, 2-undecylpentadecanol, 2-hexyldecyl alcohol, isocetyl alcohol, octyldodecanol and mixtures thereof, preferably octyldodecanol.

Non-Volatile Hydrocarbon-Based Oils with Ester Function(s)

The non-volatile hydrocarbon-based second oil can also comprise at least one ester oil other than the first oil(s).

Advantageously, the non-volatile hydrocarbon-based ester oil is chosen from hydrocarbon-based compounds comprising one or more ester functions and comprising at least one aromatic, saturated or unsaturated, linear or branched hydrocarbon-based group, the total number of carbon atoms preferably being at least 12. In addition, the ester oil can optionally comprise one or more ether or hydroxyl functions.

Among the suitable ester oils, mention may be made of mono- and diesters optionally comprising one or two ether groups; aromatic, saturated or unsaturated, linear or branched triesters optionally comprising one to three ether groups; tetraesters; polyesters obtained by condensation of an unsaturated fatty acid dimer and/or trimer and of a diol; esters and polyesters of a monocarboxylic or dicarboxylic acid and of a dimer diol or of monoalcohols; vegetable oils, and also mixtures thereof.
Aromatic, saturated or unsaturated, linear or branched mono- or diesters comprising more particularly from 12 to 80 carbon atoms and optionally comprising one or two ether groups. Mention may be made, among the compounds of this type, of monoesters or diesters obtained from a saturated or unsaturated monocarboxylic or dicarboxylic fatty acid, in particular comprising from 4 to 28 and preferably from 4 to 24 carbon atoms, optionally comprising at least one free hydroxyl, on the one hand, and from a saturated or unsaturated monoalcohol or polyol, comprising from 2 to 26 and in particular from 3 to 24 carbon atoms and from 1 to 6 hydroxyl groups, on the other hand; the number of carbon atoms being at least 12 and preferably at least 16. In addition, the ester can optionally comprise one or two ether groups and optionally one or two hydroxyl groups.

By way of example, mention may be made of 2-octyldodecyl neopentanoate, isodecyl neopentanoate, isotridecyl neopentanoate, isostearyl neopentanoate, isostearyl heptanoate, cetostearyl octanoate, cetyl octanoate, tridecyl octanoate, isononyl isononanoate, isotridecyl isononanoate, octyl isononanoate, hexyl laurate, 2-hexyldecyl laurate, 2-ethylhexyl palmitate, isopropyl palmitate, ethyl palmitate, 2-octyldecyl palmitate, isopropyl myristate, 2-octyldodecyl myristate, isopropyl stearate, butyl stearate, octyl stearate, 2-octyldodecyl stearate, glyceryl stearate, isopropyl isostearate, isostearyl isostearate, isostearyl behenate, isocetyl stearate, mixtures of esters of capric acid, of caprylic acid and of a coconut-derived alcohol (C₁₂-C₁₈ alcohols), 2-octyldodecyl erucate, oleyl erucate, isostearyl lactate, octyl hydroxystearate, octyldodecyl hydroxystearate, diisostearyl malate, isocetyl stearoyl stearate, diisostearyl adipate, or mixtures thereof.

Mention may also be made of optionally hydroxylated esters of a C₂-C₈ mono- or polycarboxylic acid and of a C₂-C₈ alcohol. Suitable in particular for the implementation of the invention are the monoesters of a C₂-C₈ carboxylic acid and of a C₂-C₈ alcohol, which are optionally hydroxylated, and the diesters of a C₂-C₈ dicarboxylic acid and of a C₂-C₈ alcohol, which are optionally hydroxylated, such as diisopropyl adipate, bis(2-ethylhexyl) adipate, dibutyl adipate or bis(2-ethylhexyl) succinate.

Mention may also be made of aromatic, unsaturated or saturated, linear or branched esters of a saturated or unsaturated monocarboxylic acid, in particular comprising from 4 to 28 carbon atoms, and of diols, in particular glycols, notably of C₂-C₅, of glycerol or of polyglycerol. Mention may be made for example of propylene glycol monoisostearate, propylene glycol monoricinoleate, neopentyl glycol dicaprate, neopentyl glycol diheptanoate, propylene glycol dioctanoate, diethylene glycol diisononanoate, polyglyceryl-2 diisostearate, polyglyceryl-3 diisostearate, ethylene glycol dibenzoate, diethylene glycol dibenzoate, propylene glycol dibenzoate, dipropylene glycol dibenzoate and also mixtures thereof.
Non-aromatic, saturated or unsaturated, linear or branched triesters comprising up to 80 carbon atoms and optionally comprising one to three ether groups. Suitable for the invention are the esters obtained from aromatic, saturated or unsaturated, linear or branched, C₂-C₄₀, preferably C₄-C₄₀, mono- or polycarboxylic acids, which are optionally hydroxylated, and from C₂-C₄₀, preferably C₃-C₄₀, polyols or monoalcohols; said polyesters optionally comprising at least one free hydroxyl.

Use may be made, for example, of triacetin and also triglycerides of saturated or unsaturated C₄-C₃₆ fatty acids, more particularly saturated or unsaturated, linear or branched C₈-C₂₀ fatty acids, such as, for example, triglycerides of heptanoic acid or octanoic acid; in particular, mention may be made of saturated triglycerides, such as caprylic/capric triglyceride, for example such as the products sold under the DUB MCT range by Stéarinerie Dubois, glyceryl triheptanoate, glyceryl trioctanoate, triglycerides of C₁₈-36 acid, such as those sold under the reference DUB TGI 24 by Stéarinerie Dubois), or glyceryl triisostearate.

Mention may also be made of triesters of glycerol or polyglycerol and of monocarboxylic acids, such as polyglycerol-2 triisostearate or glyceryl tri (2-decyltetradecanoate).

Mention may also be made, by way of example, of oils comprising three ester functions, which are optionally hydroxylated or acetylated, of a C₂-C₉ acid comprising three carboxyl functions, which is optionally hydroxylated, and of a C₂-C₂₀ monoalcohol. Mention may be made of citric acid esters, such as, for example, triethyl citrate, trioctyl citrate, tributyl citrate, tributyl acetylcitrate and likewise tridecyl trimellitate, and also mixtures thereof.
Tetraesters comprising in particular from 35 to 80 carbon atoms, optionally comprising one to three ether groups, such as tetraesters of pentaerythritol or polyglycerol and of a monocarboxylic acid, for example such as pentaerythrityl tetrapelargonate, pentaerythrityl tetraisostearate, pentaerythrityl tetraisononanoate, polyglyceryl-2 tetraisostearate or pentaerythrityl tetra(2-decyltetradecanoate).
Polyesters obtained by condensation of unsaturated fatty acid dimer and/or trimer and of diol, such as those described in patent application FR 0 853 634, in particular such as of dilinoleic acid and of 1,4-butanediol. Mention may notably be made in this respect of the polymer sold by BioSynthis under the name Viscoplast 14436H (INCI name: Dilinoleic Acid/Butanediol Copolymer) or copolymers of polyols and of diacid dimers, and esters thereof, such as Hailuscent ISDA, having the INCI name Polyglyceryl-2 Isostearate/Dimer Dilinoleate Copolymer.
Esters of a dimer diol or of a polyol, and of a mono- and/or dicarboxylic acid. Mention may be made for example of esters of a dimer diol and of a fatty acid and esters of a dimer diol and of a dicarboxylic acid dimer, in particular which may be obtained from a dicarboxylic acid dimer derived in particular from the dimerization of an unsaturated fatty acid notably of C₈ to C₃₄, notably of C₁₂ to C₂₂, in particular of C₁₆ to C₂₀ and more particularly of C₁₈, such as esters of dilinoleic diacids and of dilinoleic dimer diols, having the INCI name Dimer Dilinoleyl Dimer Dilinoleate, for instance those sold by Nippon Fine Chemical under the trade names Lusplan DD-DA5® and DD-DA7®.

Also suitable are esters derived from the esterification of a polyol, of at least one monocarboxylic acid and of at least one dicarboxylic acid, as described in particular in patent U.S. Pat. No. 7,317,068. The polyol comprises more particularly 2 to 20 carbon atoms and 2 to 8 hydroxyl groups, it preferably being pentaerythritol. More particularly, the monocarboxylic acid comprises from 4 to 30 carbon atoms, more particularly from 6 to 22 carbon atoms, such as preferably stearic acid, isostearic acid, caprylic acid, capric acid or combinations thereof; as regards the aromatic, unsaturated or saturated, linear or branched dicarboxylic acid, it comprises more particularly from 4 to 10 carbon atoms, and is preferably adipic acid. By way of example, mention may be made of pentaerythrityl isostearate/caprate/caprylate/adipate, sold in particular under the name Crodamol L by Croda, pentaerythrityl adipate/caprate/caprylate/heptanoate sold under the name Lexfeel 700 EX-LO-MB by Inolex.
Vegetable oils such as, for example, jojoba oil, sunflower oil, wheat germ oil, castor oil, maize oil, olive oil, ximenia oil, pracaxi oil, coriander oil, macadamia oil, passionflower oil, argan oil, sesame oil, grapeseed oil, avocado oil, soya oil, kernel oil (Prunus Armeniaca Kernel Oil), sweet almond oil, rapeseed oil, cottonseed oil, hazelnut oil, alfalfa oil, poppy oil, pumpkin oil, marrow oil, blackcurrant oil, evening primrose oil, millet oil, barley oil, quinoa oil, rye oil, safflower oil, candlenut oil, the liquid fraction of shea butter and the liquid fraction of cocoa butter, and also mixtures thereof;

The content of polar non-volatile hydrocarbon-based second oil(s) advantageously ranges from 8% to 20% by weight, advantageously from 8% to less than 20% by weight and more particularly from 9% to 15% by weight, relative to the total weight of the composition.

According to one advantageous variant of the invention, the composition comprises at least one C₁₀-C₂₆ alcohol as described above in a content ranging from 8% to 20% by weight and more particularly from 9% to 15% by weight, relative to the total weight of the composition.

More particularly, the (ethylcellulose/(ethylcellulose+C₁₀-C₂₆ alcohol(s)) weight ratio*100 is at least 10%, more particularly at least 15% and advantageously does not exceed 60%, preferably does not exceed 50%.

### Non-Volatile Silicone Third Oils

The composition according to the invention comprises at least a third oil which is chosen from non-volatile phenylated silicones comprising at least one dimethicone fragment.

For the purposes of the invention, the term “silicone oil” means an oil comprising at least one silicon atom, and notably at least one Si—O group.

The term “phenylated” specifies that said oil comprises, in its structure, at least one phenyl radical.

The term “dimethicone fragment” denotes a divalent siloxane group, the silicon atom of which bears two methyl radicals, this group not being located at an or at the ends of the molecule. It may be represented by the following formula:

(Si(CH₃)₂—O)—.

Preferably, the silicones do not comprise a C₂-C₃ alkylene oxide group or a glycerolated group.

Preferably the composition comprises at least a third oil which is chosen from the compounds having the following INCI names: Trimethylsiloxyphenyl Dimethicone, Diphenyl Dimethicone, Tetramethyl Tetraphenyl Trisiloxane and also mixtures thereof, preferably Trimethylsiloxyphenyl Dimethicone.

The Diphenyl Dimethicones are in particular sold by Shin Etsu under the names KF-54, KF54HV, KF-50-300CS, KF-53 d and KF-50-100CS. The Trimethylsiloxy Phenyl Dimethicones are for example sold by Wacker Chemie under the names Belsil PDM 1000 and Belsil PDM 20.

The composition according to the invention comprises more particularly from 20% to 40% by weight and preferably from 25% to 35% by weight relative to the total weight of the composition.

### Optional Non-Volatile Silicone Fourth Oils

According to one variant of the invention, the composition comprises at least a nonvolatile silicone fourth oil which is phenylated with no dimethicone fragment, or is non-phenylated.

Representative examples of these non-volatile non-phenylated silicone oils that may be mentioned include polydimethylsiloxanes and alkyl dimethicones.

It should be noted that “dimethicone” (INCI name) corresponds to a polydimethylsiloxane (chemical name).

Preferably, these non-volatile, non-phenylated silicone oils are chosen from polydimethylsiloxanes and alkyl dimethicones comprising at least one C₂-C₂₄ alkyl group, and also mixtures thereof.

With regard to the non-volatile phenylated silicone oils with no dimethicone fragment, mention may be made of the compounds having the following INCI names: Phenyltrimethicone, Trimethyl Pentaphenyl Trisiloxanes, alone or as mixtures.

Thus, these oils can be chosen from dimethicone, cetyl dimethicone, stearyl dimethicone, phenyltrimethicone, trimethyl pentaphenyl trisiloxane, and diphenylsiloxy phenyl trimethicone, alone or as mixtures.

As non-volatile, non-phenylated silicone oils suitable for the implementation of the invention, mention may be made of those sold by Wacker under the Belsil DM range, by Dow Corning with the Xiameter PMX 200 Silicone Fluid range, and by Shin Etsu with the KF-96 A range.

The alkyl dimethicones may be sold for example under the trade references Abil Wax 9800 and Abil Wax 9801 from Evonik Goldschmidt, or else Dowsil 2502 Cosmetic Fluid and Dowsil 2503 Cosmetic Wax from Dow Corning; and mixtures thereof.

The phenylated silicones are in particular sold under the names PH-1555 HRI Cosmetic Fluid (trimethyl pentaphenyl trisiloxane) and Dow Corning 556 Cosmetic Grade Fluid (phenyl trimethicone) by Dow Corning; diphenyl dimethicones such as the products KF-54, KF54HV, KF-50-300CS, KF-53 d and KF-50-100CS or Diphenylsiloxy Phenyl Trimethicone KF56 A sold by Shin-Etsu.

The content of non-volatile silicone fourth oil(s) which are phenylated with no dimethicone fragment or are non-phenylated, if the composition comprises same, meets the requirement relating to the oil 1, oil 2 and oil 4/oil 3 weight ratio of less than or equal to 1, mentioned previously.

Advantageously, the content of fourth oil(s), if the composition comprises same, is less than or equal to 5% by weight and more particularly less than or equal to 1% by weight, relative to the total weight of the composition.

### Non-Polar, Non-Volatile Hydrocarbon-Based Oils

The composition according to the invention may optionally comprise at least one non-polar, non-volatile hydrocarbon-based oil.

The term “non-polar oil” is intended to mean, for the purposes of the present invention, an oil chosen from hydrocarbons, that is to say from compounds comprising only carbon and hydrogen atoms.

Preferably, the non-polar, non-volatile hydrocarbon-based oil can be chosen from linear or branched hydrocarbons of mineral, vegetable or synthetic origin, such as, for example:
liquid paraffin,
squalane, in particular of vegetable origin,
isoeicosane,
mixtures of saturated linear hydrocarbons, more particularly of C₁₅-C₂₈, such as the mixtures of which the INCI names are, for example, the following: C15-19 Alkane, C18-21 Alkane, C21-28 Alkane, for example the products Gemseal 40, Gemseal 60, Gemseal 120 sold by Total, Emogreen L19 sold by SEPPIC,
hydrogenated or non-hydrogenated polybutenes, for instance products of the Indopol range sold by Ineos Oligomers,
hydrogenated or non-hydrogenated polyisobutenes, for instance the non-volatile compounds of the Parleam® range sold by Nippon Oil & Fats,
hydrogenated or non-hydrogenated polydecenes, for instance non-volatile compounds of the Silkflo range sold by Ineos, Dekanex by IMCD,
and mixtures thereof.

The content of non-polar, non-volatile hydrocarbon-based oil(s), if the composition comprises same, represents from 0.1% to 15% by weight and more particularly from 1% to 12% by weight, relative to the total weight of the composition.

### Volatile Oils

According to one particular embodiment of the invention, the composition may also comprise at least one volatile oil.

The volatile oil can in particular be a silicone oil, a hydrocarbon-based oil, which is preferably non-polar, and also mixtures thereof.

The term “volatile” is understood to mean an oil, the vapour pressure at 25 °C and atmospheric pressure of which is between 2.66 Pa and 40,000 Pa and preferably between 2.66 and 1300 Pa.

As volatile silicone oils that may be used in the invention, mention may be made of linear or cyclic silicones with a viscosity at ambient temperature of less than 8 centistokes (cSt) (8×10⁻⁶ m²/s), and in particular containing from 2 to 10 silicon atoms and in particular from 2 to 7 silicon atoms, these silicones optionally comprising alkyl or alkoxy groups containing from 1 to 10 carbon atoms.

As volatile silicone oils that may be used in the invention, mention may notably be made of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Among the volatile hydrocarbon-based oils, which are preferably non-polar, mention may be made of volatile hydrocarbon-based oils containing from 8 to 16 carbon atoms, and mixtures thereof, and in particular:
branched C₈-C₁₆ alkanes, such as C₈-C₁₆ isoalkanes (also known as isoparaffins), isododecane, isodecane or isohexadecane, and for example the oils sold under the trade names Isopar, which are sold by ExxonMobil,
linear alkanes, for instance n-dodecane (C12) and n-tetradecane (C14) sold by Sasol under the respective references Parafol 12-97 and Parafol 14-97, the undecanetridecane mixture (Cetiol UT), the mixtures of n-undecane (C11) and of n-tridecane (C13) obtained in Examples 1 and 2 of patent application WO 2008/155059 from Cognis, and mixtures thereof.

When the composition comprises at least one additional volatile oil, their content more particularly represents from 0.1% to 15% by weight and more particularly from 1% to 12% by weight, relative to the total weight of the composition.

### Waxes

The composition according to the invention may advantageously comprise at least one wax.

For the purposes of the present invention, the term “wax” means a lipophilic compound, which is solid at ambient temperature (25 °C), with a reversible solid/liquid change of state, which has a melting point of greater than or equal to 30 °C that may be up to 120 °C

For the purposes of the invention, the melting point corresponds to the temperature of the most endothermic peak observed on thermal analysis (DSC) as described in the standard ISO 11357-3; 1999. The melting point of the wax can be measured using a differential scanning calorimeter (DSC), for example the calorimeter sold under the name DSC Q2000 by TA Instruments with the TA Universal Analysis software. The measuring protocol is as follows:

A sample of 5 mg of wax placed in a crucible is subjected to a first temperature rise ranging from −20 °C to 120 °C, at a heating rate of 10 °C/minute, it is then cooled from 120 °C to −20 °C at a cooling rate of 10 °C/minute and is finally subjected to a second temperature rise ranging from −20 °C to 120 °C at a heating rate of 5 °C/minute.

During the second temperature rise, the melting point of the solid fatty substance is measured, which corresponds to the temperature of the most endothermic peak observed of the melting curve, representing the variation in the difference in power absorbed as a function of the temperature.

The enthalpy of fusion of the wax (ΔHf), corresponding to the integral of the entire melting curve obtained, can also be measured. This enthalpy of fusion of the wax is the amount of energy required to cause the compound to change from the solid state to the liquid state. It is expressed in J/g.

The waxes may be silicone, and preferably hydrocarbon-based, waxes. They are also of vegetable, mineral, animal and/or synthetic origin.

In particular, the waxes have a melting point of preferably greater than or equal to 35 °C and better still greater than or equal to 40 °C

### Non-Polar Waxes

For the purposes of the present invention, the term “non-polar hydrocarbon-based wax” is intended to mean a wax constituted only of carbon and hydrogen atoms and free of heteroatoms, such as for example N, O, Si, P, etc.

Mention may in particular be made, by way of examples of non-polar waxes suitable for the invention, of hydrocarbon waxes, for instance microcrystalline waxes, paraffin waxes, ozokerite, polymethylene waxes, polyethylene waxes and microwaxes, in particular of polyethylene.

### Polar Waxes

The polar waxes may notably be hydrocarbon-based or silicone waxes.

For the purposes of the present invention, the term “polar hydrocarbon-based wax” is intended to mean a wax of which the chemical structure is formed essentially of, or even constituted of, carbon and hydrogen atoms, and which comprises at least one heteroatom more particularly chosen from oxygen, optionally nitrogen, or mixtures thereof. It may thus contain alcohol, ester, ether, carboxylic acid, amine and/or amide groups.

The term “silicone wax” means an oil comprising at least one silicon atom and notably comprising Si—O groups.

According to a first preferred embodiment, the polar wax is a hydrocarbon-based wax.

A wax chosen from ester waxes and alcohol waxes is in particular preferred as polar hydrocarbon-based wax.

According to the invention, the term “ester wax” means a wax comprising at least one ester function. The ester waxes may also be hydroxylated.

The expression “alcohol wax” is understood according to the invention to mean a wax comprising at least one alcohol function, i.e. comprising at least one free hydroxyl (OH) group.

Use may notably be made, as ester wax, of, alone or as mixtures:
i) waxes of formula R₁COOR₂ in which R₁ and R₂ represent linear, branched or cyclic aliphatic chains, the number of atoms of which ranges from 6 to 50, in particular from 10 to 50, which may contain a heteroatom such as for example O or N, and the melting point of which ranges more particularly from 30 °C to 120 °C In particular, use may be made, as an ester wax, of a C₂₀-C₄₀ alkyl (hydroxystearyloxy) stearate (the alkyl group comprising from 20 to 40 carbon atoms), alone or as a mixture, or a C₂₀-C₄₀ alkyl stearate. Such waxes are sold in particular under the names Kester Wax K 82 P®, Hydroxypolyester K 82 P®, Kester Wax K 80 P® or Kester Wax K82H by Koster Keunen. Use may also be made of stearyl heptanoate and stearyl caprylate and mixtures thereof;
ii) di(1,1,1-trimethylolpropane) tetrastearate;
iii) diester waxes of a dicarboxylic acid, of general formula
R³—(—OCO—R⁴—COO—R⁵), in which R³ and R⁵ are identical or different, preferably identical, and represent a C₄-C₃₀ alkyl group and R⁴ represents a linear or branched C₄-C₃₀ aliphatic group which may or may not contain one or more unsaturations. Preferably, the C₄-C₃₀ aliphatic group is linear and unsaturated;
iv) mention may also be made of the waxes obtained by catalytic hydrogenation of animal or vegetable oils having in particular linear or branched C₈-C₃₂ fatty chains, for example such as hydrogenated jojoba oil, hydrogenated sunflower oil, hydrogenated castor oil or hydrogenated coconut oil, and also the waxes obtained by hydrogenation of castor oil esterified with cetyl alcohol, such as those sold in the Phytowax Castor range, for example Phytowax Castor 22L73®, or else the waxes obtained by hydrogenation of olive oil esterified with stearyl alcohol, such as those of the Phytowax Olive range, for example Phytowax Olive 18L57, sold by Sophim. Such waxes are described in particular in application FR2792190;
v) the waxes corresponding to the partial or total esters, preferably total esters, of a saturated, optionally hydroxylated, C₁₆-C₃₀ carboxylic acid with glycerol. The term “total esters” is intended to mean that all the hydroxyl functions of glycerol are esterified. Mention may be made, by way of example, of trihydroxystearin (or glyceryl trihydroxystearate), tristearin (or glyceryl tristearate) or tribehenin (or glyceryl tribehenate), alone or as a mixture. Among the suitable compounds, mention may be made of triesters of glycerol and of 12-hydroxystearic acid, or hydrogenated castor oil, for instance Thixcin R and Thixcin E sold by Elementis Specialties;
vi) Mention may also be made of waxes of animal or vegetable origin, such as beeswax, synthetic beeswax, carnauba wax, candelilla wax, rice bran wax, ouricury wax, esparto grass wax, cork fibre wax, sugar cane wax, Japan wax, sumac wax, montan wax, orange wax, laurel wax, or sunflower wax, in particular refined sunflower wax;
vii) mention may also be made of natural or synthetic polyoxyalkylenated or polyglycerolated hydrocarbon-based waxes, of animal or vegetable origin; the number of (C₂-C₄) oxyalkylene units can range from 2 to 100, the number of glycerol units can range from 1 to 20. By way of examples, mention may be made of polyoxyethylenated beeswaxes, such as PEG-6 beeswax or PEG-8 beeswax; polyoxyethylenated carnauba waxes, such as PEG-12 carnauba; polyoxyethylenated or polyoxypropylenated and hydrogenated or non-hydrogenated lanolin waxes, such as PEG-30 lanolin or PEG-75 lanolin; PPG-5 lanolin wax glyceride; polyglycerolated beeswaxes, in particular polyglyceryl-3 beeswax, the Acacia decurrens/Jojoba/Sunflower Seed Wax/Polyglyceryl-3 Esters mixture, polyglycerolated vegetable waxes, such as mimosa, jojoba or sunflower waxes, and mixtures thereof (Acacia decurrens/Jojoba/Sunflower Seed Wax Polyglyceryl-3 Esters.

According to another embodiment, the polar wax may be an alcohol wax. Mention may be made, as alcohol wax, of mixtures of saturated linear C₃₀-C₅₀ alcohols, such as, for example, the wax Performacol 550 Alcohol from New Phase Technology, stearyl alcohol and cetyl alcohol, or mixtures thereof.

### Silicone Waxes

Mention may be made, as silicone wax, for example, of the mixtures comprising a compound of C30-45 Alkyldimethylsilyl Polypropylsilsesquioxane (INCI name) type, for example the product Dow Corning SW-8005 C30 Resin Wax sold by Dow Corning. Mention may also be made of the mixtures comprising a compound of the C30-45 Alkyl Methicone (INCI name) type, such as, for example, the product Dow Corning® AMS-C30 Cosmetic Wax. Mention may also be made of siliconized beeswax.

Preferably, the wax, if the composition comprises same, is chosen from hydrocarbon-based waxes. More particularly, it is chosen from non-polar waxes; polar hydrocarbon-based waxes such as waxes of animal or vegetable origin, waxes of animal or vegetable origin obtained by catalytic hydrogenation of animal or vegetable oils; alcohol waxes; and also mixtures thereof; and preferably from non-polar hydrocarbon-based waxes, alone or as mixtures.

The wax content, if the composition comprises same, advantageously ranges from 1% to 20% by weight, in particular from 5% to 15% by weight, relative to the total weight of the composition.

### Pasty Compounds

The composition according to the invention can also comprise at least one compound which is pasty at ambient temperature and atmospheric pressure.

For the purposes of the present invention, the term “pasty” means a lipophilic compound with a reversible solid/liquid change of state, having notably in the solid state an anisotropic crystal organization, and comprising at ambient temperature a liquid fraction and a solid fraction.

In other words, the starting melting point of the pasty compound may be lower than ambient temperature. The liquid fraction of the pasty compound, measured at ambient temperature, can represent from 9% to 97% by weight of the pasty compound. This fraction that is liquid at ambient temperature preferably represents between 15% and 85%, more preferably between 40% and 85%, by weight.

The melting point of the pasty fatty substance is determined according to the same principle as that described in detail above for the waxes.

In the case of a pasty compound, the measurement protocol is, however, as follows:

A sample of 5 mg of pasty fatty substance placed in a crucible is subjected to a first temperature rise passing from −20 °C to 100 °C, at a heating rate of 10 °C/minute, it is then cooled from 100 °C to −20 °C at a cooling rate of 10 °C/minute and it is finally subjected to a second temperature rise passing from −20 °C to 100 °C at a heating rate of 5 °C/minute.

The melting point of the pasty fatty substance is the value of the temperature corresponding to the top of the peak on the curve representing the variation in the difference in power absorbed as a function of the temperature.

It should be noted that the liquid fraction by weight of the pasty fatty substance at ambient temperature is equal to the ratio of the heat of fusion consumed at ambient temperature to the heat of fusion of the pasty fatty substance.

The heat of fusion of the pasty fatty substance is the heat consumed by said substance in order to pass from the solid state to the liquid state. The pasty fatty substance is said to be in the solid state when all of its mass is in crystalline solid form. The pasty fatty substance is said to be in the liquid state when all of its mass is in liquid form.

The heat of fusion of the pasty fatty substance is the amount of energy required to make the pasty fatty substance change from the solid state to the liquid state. It is expressed in J/g. The heat of fusion of the pasty fatty substance is equal to the area under the curve of the thermogram obtained.

The pasty compound can in particular be chosen from synthetic pasty compounds and fatty substances of vegetable origin.

The pasty compound or compounds can be chosen in particular from:
lanolin and derivatives thereof, such as lanolin alcohol, oxyethylenated lanolins, acetylated lanolin, lanolin esters such as isopropyl lanolate, and oxypropylenated lanolins,
petroleum jelly (also known as petrolatum),
ethers of pentaerythritol and of C₂-C₄ polyalkylene glycol, for example the compounds with the INCI names below: PEG-5 Pentaerythrityl Ether, PPG-5 Pentaerythrityl Ether, and mixtures thereof. Mention may be made for example of the mixture sold under the name Lanolide by Vevy,
liposoluble polyethers resulting from the polyetherification between one or more C₂-C₁₀₀ and preferably C₂-C₅₀ diols. Consideration is given in particular, among the liposoluble polyethers, to copolymers of ethylene oxide and/or of propylene oxide with long-chain C₆-C₃₀ alkylene oxides, more preferably such that the ratio by weight of the ethylene oxide and/or propylene oxide to alkylene oxides in the copolymer is from 5:95 to 70:30. In this family, mention will be made in particular of the product with the INCI name PEG-45/Dodecyl Glycol Copolymer sold for example under the brand name Elfacos ST9 by Akzo Nobel,
esters resulting from the condensation of a preferably saturated, linear or branched, C₆-C₁₀ dicarboxylic acid and of an ester of diglycerol and of optionally hydroxylated, preferably saturated, linear or branched, C₆-C₂₀ monocarboxylic acids, in particular the diester obtained by condensation of adipic acid and of a mixture of esters of diglycerol with a mixture of C₆-C₂₀ fatty acids, such as caprylic acid, capric acid, stearic acid, isostearic acid and 12-hydroxystearic acid, sold in particular under the reference Softisan® 649 by Cremer Oleo (INCI name: Bis-Diglyceryl Polyacyladipate-2),
triglycerides of fatty acids which are saturated or unsaturated, linear or branched, optionally mono- or polyhydroxylated, preferably C₁₂-C₁₈, and optionally hydrogenated (completely or partially); such as, for example, the glycerides of saturated C₁₂-C₁₈ fatty acids sold under the name Softisan 100® by Cremer Oleo (INCI name: Hydrogenated Coco-Glycerides),
esters of a dimer diol, or of polyol, and of a dimer diacid, such as, for example:
esters of a dimer dilinoleyl alcohol and of dilinoleic acid, the hydroxyl groups of which are esterified by a mixture of phytosterols, of behenyl alcohol and of isostearyl alcohol, for example the ester sold under the name Plandool G by Nippon Fine Chemical (INCI name: Bis-Behenyl/Isostearyl/Phytosteryl Dimer Dilinoleyl Dimer Dilinoleate);
esters of dilinoleic acid and of a mixture of phytosterols, of isostearyl alcohol, of cetyl alcohol, of stearyl alcohol and of behenyl alcohol, for example the ester sold under the name Plandool H or Plandool S by Nippon Fine Chemical (INCI name: Phytosteryl/Isostearyl/Cetyl/Stearyl/Behenyl Dimer Dilinoleate);
butters of vegetable origin, such as mango butter, such as the product sold under the reference Lipex 203 by AarhusKarlshamn, shea butter, in particular the product of which the INCI name is Butyrospermum Parkii Butter, such as the product sold under the reference Sheasoft® by AarhusKarlshamn, cupuacu butter (Rain Forest RF3410 from Beraca Sabara), murumuru butter (Rain Forest RF3710 from Beraca Sabara), cocoa butter; and also orange wax, for instance the product sold under the reference Orange Peel Wax by Koster Keunen,
completely or partially hydrogenated vegetable oils, such as, for example, hydrogenated soybean oil, hydrogenated coconut oil, hydrogenated rapeseed oil, mixtures of hydrogenated vegetable oils, such as the mixture of hydrogenated soybean, coconut, palm and rapeseed vegetable oil, for example the mixture sold under the reference Akogel® by AarhusKarlshamn (INCI name: Hydrogenated Vegetable Oil), the trans-isomerized partially hydrogenated jojoba oil manufactured or sold by Desert Whale under the commercial reference Iso-Jojoba-50®, and partially hydrogenated olive oil, such as, for example, the compound sold under the reference Beurrolive by Soliance,
hydrogenated castor oil esters, such as hydrogenated castor oil dimer dilinoleate, for example Risocast-DA-L sold by Kokyu Alcohol Kogyo, and hydrogenated castor oil isostearate, for example Salacos HCIS (V-L) sold by Nisshin Oil,
and mixtures thereof.

Preferably, the pasty compound(s) is/are chosen from the products with the INCI names below: PEG-45/Dodecyl Glycol Copolymer, Bis-Diglyceryl Polyacyladipate-2, Hydrogenated Coco-Glycerides, from esters of a dimer diol, or of a polyol, and of a diacid dimer, from butters of vegetable origin, and mixtures thereof.

If the composition comprises at least one pasty compound, its/their content ranges from 0.5% to 15% by weight and preferably from 2% to 12% by weight, relative to the total weight of the composition.

### Colorants

The composition according to the invention preferably comprises at least one colorant, which is synthetic, natural or of natural origin.

More particularly, the content of colorant(s) in the composition is advantageously at least 0.01% by weight, more particularly between 0.05% and 30% by weight, in particular between 0.1% and 25% by weight, preferably from 0.1% to 15% by weight, relative to the total weight of the composition.

The colorant(s) may be chosen from coated or uncoated pigments, water-soluble dyes, liposoluble dyes, and mixtures thereof.

### Pigments

The term “pigments” means white or coloured, mineral or organic particles, which are insoluble in the medium of the composition, and which are intended to colour and/or opacify the resulting composition and/or deposit. Therefore, the pigments are not fillers.

According to a first particular embodiment, the pigments used are chosen from mineral pigments.

The term “mineral pigment” refers to any pigment that satisfies the definition in Ullmann's encyclopaedia in the chapter on inorganic pigments. Among the mineral pigments that are useful in the present invention, mention may be made of zirconium oxide or cerium oxide, and also zinc oxide, iron oxide (black, yellow or red) or chromium oxide, manganese violet, ultramarine blue, chromium hydrate and ferric blue, titanium dioxide, and metal powders, for instance aluminium powder or copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The size of the pigment that is useful in the context of the present invention is generally greater than 100 nm and may range up to 10 μm, preferably from 200 nm to 5 μm and more preferentially from 300 nm to 1 μm.

According to one particular form of the invention, the pigments have a size characterized by a D[50] of greater than 100 nm and possibly ranging up to 10 μm, preferably from 200 nm to 5 μm and more preferentially from 300 nm to 1 μm.

The sizes are measured by static light scattering using a commercial MasterSizer 3000® particle size analyser from Malvern, which makes it possible to determine the particle size distribution of all of the particles over a wide range which may extend from 0.01 μm to 1000 μm. The data are processed on the basis of the standard Mie scattering theory. This theory is the most suitable for size distributions ranging from submicron to multimicron; it allows an “effective” particle diameter to be determined. This theory is notably described in the publication by Van de Hulst, H. C., Light Scattering by Small Particles, Chapters 9 and 10, Wiley, New York, 1957.

D[50] represents the maximum size exhibited by 50% by volume of the particles.

In the context of the present invention, the mineral pigments are more particularly iron oxide and/or titanium dioxide. Examples that may be mentioned more particularly include titanium dioxides and iron oxide coated with aluminium stearoyl glutamate, sold, for example, under the reference NAI® by Miyoshi Kasei.

As mineral pigments that may be used in the invention, mention may also be made of pearlescent agents.

The term “pearlescent agents” should be understood as meaning coloured particles of any form, which may or may not be iridescent, notably produced by certain molluscs in their shell, or alternatively synthesized, and which have a colour effect via optical interference.

The pearlescent agents may be chosen from pearlescent pigments such as titanium mica coated with an iron oxide, titanium mica coated with bismuth oxychloride, titanium mica coated with chromium oxide, titanium mica coated with an organic dye and also pearlescent pigments based on bismuth oxychloride. They may also be mica particles, at the surface of which are superposed at least two successive layers of metal oxides and/or of organic colorants.

Examples of pearlescent agents that may also be mentioned include natural mica covered with titanium oxide, with iron oxide, with natural pigment or with bismuth oxychloride.

The pearlescent agents can more particularly have a yellow, pink, red, bronze, orangey, brown, gold and/or coppery colour or tint.

Among the pigments that may be used according to the invention, mention may also be made of those having an optical effect different from a simple conventional colouring effect, i.e. a unified and stabilized effect such as produced by conventional colorants, for instance monochromatic pigments. For the purposes of the invention, the term “stabilized” means lacking the effect of variability of the colour with the angle of observation or in response to a temperature change.

For example, this material may be chosen from particles with a metallic tint, goniochromatic colouring agents, diffractive pigments, thermochromic agents, optical brighteners, and also fibres, notably interference fibres. Needless to say, these various materials may be combined in order simultaneously to afford two effects, or even a novel effect in accordance with the invention.

According to one particular embodiment, the composition according to the invention comprises at least one uncoated pigment.

According to another particular embodiment, the composition according to the invention comprises at least one pigment coated by at least one lipophilic or hydrophobic compound.

This type of pigment is particularly advantageous. Insofar as they are treated with a hydrophobic compound, they show predominant affinity for an oily phase, which can then convey them.

The coating may also comprise at least one additional non-lipophilic compound.

For the purposes of the invention, the “coating” of a pigment according to the invention generally denotes the total or partial surface treatment of the pigment with a surface agent, absorbed, adsorbed or grafted onto said pigment.

The surface-treated pigments may be prepared according to surface treatment techniques of chemical, electronic, mechanochemical or mechanical nature that are well known to a person skilled in the art. Commercial products may also be used.

The surface agent may be absorbed, adsorbed or grafted onto the pigments by evaporation of solvent, chemical reaction and creation of a covalent bond.

According to one variant, the surface treatment consists in coating the pigments.

The coating may represent from 0.1% to 20% by weight and in particular from 0.5% to 5% by weight relative to the total weight of the coated pigment.

The coating may be produced, for example, by adsorption of a liquid surface agent onto the surface of the solid particles by simple mixing with stirring of the particles and of said surface agent, optionally with heating, prior to the incorporation of the particles into the other ingredients of the makeup or care composition.

The coating may be produced, for example, by chemical reaction of a surface agent with the surface of the solid pigment particles and creation of a covalent bond between the surface agent and the particles. This method is notably described in patent U.S. Pat. No. 4,578,266.

The chemical surface treatment may consist in diluting the surface agent in a volatile solvent, dispersing the pigments in this mixture and then slowly evaporating off the volatile solvent, so that the surface agent is deposited on the surface of the pigments.

When the pigment comprises a lipophilic or hydrophobic coating, it is preferably present in the fatty phase of the composition according to the invention.

According to one particular embodiment of the invention, the pigments may be coated according to the invention with at least one compound chosen from silicone surface agents; fluoro surface agents; fluorosilicone surface agents; metal soaps; N-acylamino acids or salts thereof; lecithin and derivatives thereof; isopropyl triisostearyl titanate; isostearyl sebacate; natural vegetable or animal waxes; polar synthetic waxes; fatty esters; phospholipids; and mixtures thereof.

According to one particular embodiment of the invention, the pigments may be coated with a hydrophilic compound.

According to another particular embodiment, the colorant is an organic pigment, which is synthetic, natural or of natural origin.

The term “organic pigment” refers to any pigment that satisfies the definition in Ullmann's Encyclopedia in the chapter on organic pigments. The organic pigment may notably be chosen from nitroso, nitro, azo, xanthene, quinoline, anthraquinone, phthalocyanine, metal-complex type, isoindolinone, isoindoline, quinacridone, perinone, perylene, diketopyrrolopyrrole, thioindigo, dioxazine, triphenylmethane or quinophthalone compounds.

The organic pigment(s) may be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as described in patent FR 2 679 771.

The pigments may also be in the form of composite pigments as described in patent EP 1 184 426. These composite pigments may notably be composed of particles including a mineral core at least partially covered with an organic pigment and at least one binder for fixing the organic pigments to the core.

The pigment may also be a lake. The term “lake” means insolubilized dyes adsorbed onto insoluble particles, the assembly thus obtained remaining insoluble during use.

The mineral substrates onto which the dyes are adsorbed are, for example, alumina, silica, calcium sodium borosilicate or calcium aluminium borosilicate and aluminium.

Among the organic dyes, mention may be made of cochineal carmine. Mention may also be made of the products known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 4 (CI 15 510), D & C Red 33 (CI 17 200), D & C Yellow 5 (CI 19 140), D & C Yellow 6 (CI 15 985), D & C Green 5 (CI 61 570), D & C Yellow 10 (CI 77 002), D & C Green 3 (CI 42 053), D & C Blue 1 (CI 42 090).

An example of a lake that may be mentioned is the product known under the name D & C Red 7 (CI 15 850:1).

The pigment(s) is/are preferably present in contents of at least 0.01% by weight, more particularly of at least 1% by weight, and even more particularly of at least 2% by weight, relative to the weight of the composition concerned. More particularly, the colorant content is less than 30% by weight, more particularly between 0.05% and 30% by weight, and better still from 0.1% to 25% by weight, relative to the total weight of the composition.

### Water-Soluble or Liposoluble Dyes

According to another particular embodiment of the invention, the colorant is a water-soluble dye or a liposoluble dye.

For the purposes of the invention, the term “water-soluble colorant” means any natural or synthetic, generally organic compound, which is soluble in an aqueous phase or water-miscible solvents and which is capable of colouring.

For the purposes of the invention, the term “liposoluble colorant” means any natural or synthetic, generally organic compound, which is soluble in an oily phase or in solvents that are miscible with the oily phase, and which is capable of imparting colour.

As water-soluble dyes that are suitable for use in the invention, mention may notably be made of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5 and FDC Blue 1.

Among the natural water-soluble dyes, mention may be made of anthocyanins.

As liposoluble dyes that are suitable for use in the invention, mention may notably be made, for instance, of the liposoluble dyes DC Red 17, DC Red 21, DC Red 27, DC Green 6, DC Yellow 11, DC Violet 2, DC Orange 5, Sudan red and Sudan brown.

As illustrations of natural liposoluble dyes, mention may be made particularly of carotenes, for instance β-carotene, α-carotene and lycopene; quinoline yellow; xanthophylls such as astaxanthin, antheraxanthin, citranaxanthin, cryptoxanthin, canthaxanthin, diatoxanthin, flavoxanthin, fucoxanthin, lutein, rhodoxanthin, rubixanthin, siphonaxanthin, violaxanthin, zeaxanthin; annatto; curcumin; quinizarin (Ceres Green B B, D & C Green No. 6, CI 61565, 1,4-di-p-toluidinoanthraquinone, Green No. 202, quinzaine green SS) and chlorophylls.

The water-soluble or liposoluble dye(s), if the composition comprises same, is/are preferably present in contents of less than 4% by weight, or even less than 2% by weight, more preferentially ranging from 0.01% to 2% by weight and even better still from 0.02% to 1.5% by weight, relative to the total weight of the composition.

### Fillers

The composition according to the invention may also comprise at least one filler.

“Filler” denotes a particle of organic or inorganic nature which is colourless or white, which is solid, which has any shape and which is insoluble in the medium of the composition at ambient temperature and atmospheric pressure. These fillers are advantageously dispersed in the composition.

The term “inorganic” is understood to mean any compound, the chemical structure of which does not comprise a carbon atom.

The fillers may or may not be surface-coated, and in particular they can be surface-treated with silicones, amino acids, fluorinated derivatives or any other substance which promotes the dispersion and compatibility of the filler in the composition.

Such fillers are distinct from the mineral thickeners and also from the colouring agents described above.

The fillers can be spherical, that is to say comprise at least a rounded general portion, preferably defining at least a sphere portion, preferably internally defining a concavity or a hollow (sphere, globules, bowls, horseshoe, and the like), or lamellar.

Such fillers are advantageously chosen from:
silica powders, such as the porous silica microspheres sold under the name Silica Beads SB-700 by Miyoshi or Sunsphere® H-51 or Sunsphere® H-33 by Asahi Glass; or the polydimethylsiloxane-coated amorphous silica microspheres sold under the name SA Sunsphere® H-33 or SA Sunsphere® H-53 by Asahi Glass,
powders of acrylic (co) polymers and derivatives thereof, in particular:
the polymethyl methacrylate powder sold under the names Covabead® LH85 by Sensient or Microsphere M-100® by Matsumoto Yushi-Seiyaku,
the polymethyl methacrylate/ethylene glycol dimethacrylate powder sold under the name Dow Corning 5640 Microsponge® Skin Oil Adsorber by Dow Corning or Ganzpearl® GMP-0820 by Ganz Chemical,
the polyallyl methacrylate/ethylene glycol dimethacrylate powder sold under the name Poly-Pore® L200 or Poly-Pore® E200 by Amcol Health and Beauty Solutions Inc.,
the ethylene glycol dimethacrylate/lauryl methacrylate copolymer powder sold under the name Polytrap® 6603 Adsorber by Amcol Health and Beauty Solutions Inc.,
the optionally crosslinked acrylate/alkyl acrylate copolymer crosslinked acrylate/ethylhexyl acrylate copolymer powder sold under the name Techpolymer ACP-8C by Sekisui Plastics,
ethylene/acrylate copolymer powder, such as that sold under the name Flobeads® by Sumitomo Seika Chemicals,
the expanded hollow particles of acrylonitrile (co) polymer sold under the name Expancel by Akzo Nobel, or the microspheres sold under the name Micropearl F 80 ED® by Matsumoto,
polyurethane powders, for example sold under the names D-400, CS-400 and D800 by Toshiki,
silicone powders advantageously chosen from:
polymethylsilsesquioxane powders, in particular those sold under the name Tospearl, in particular Tospearl 145 A, by Momentive Performance Materials,
organopolysiloxane elastomer powders coated with silicone resin, in particular with silsesquioxane resin, such as the products sold under the name KSP-100, KSP-101, KSP-102, KSP-103, KSP-104 or KSP-105 by Shin-Etsu (INCI name: vinyl dimethicone/methicone silsesquioxane crosspolymer),
powders of silicone elastomers, such as the products sold under the name Dowsil Trefil E-506 S Silicone Powder or Trefil® Powder E-506C by Dow Corning,
powders of organosilicone particles, for example, in the form of bowls, such as those described in JP-2003 128 788 or JP-A-2000-191789 or also in application EP 1 579 841 and sold in particular by Takemoto Oil & Fat,
polyamide powders, such as Nylon® powders, in particular Nylon 12 powders, such as the nylon powders sold under the name Orgasol® 2002 EXS NAT COS by Arkema,
powders of natural organic materials, such as polysaccharide powders and in particular starch powders, especially crosslinked or non-crosslinked corn, wheat or rice starch powders, powders of starch crosslinked with octenylsuccinic anhydride sold under the name Dry-Flo® by National Starch or powders of waxy corn starch, such as those which are sold under the names C*Gel 04201 by Cargill, Corn Starch B by Roquette and Organic Corn Starch by Draco Natural Products,
spherical cellulose microparticles, such as Cellulobeads D-10, Cellulobeads D-5 and Cellulobeads USF, sold by Daito Kasei Kogyo,
particles of N—(C₈-C₂₂ acylated) amino acids; the amino acid can, for example, be lysine, glutamic acid or alanine, preferably lysine, for example Amihope LL from Ajinomoto or also that which is sold under the name Corum 5105 S by Corum,
Perlite powders, such as those sold by World Minerals under the trade name Perlite P1430, Perlite P2550, Perlite P2040 or OpTiMat® 1430 OR or 2550 OR. Europerl EMP-2 and Europerl 1 by Imerys,
zeolites, such as the products sold by Zeochem under the names Zeoflair 300, Zeoflair 200, Zeoflair 100, X-Mol and X-Mol MT,
calcium magnesium carbonate particles, such as those sold by Imerys under the name Calcidol, by LCW (Sensient) under the name Carbomat or by Omya under the name Omyacare S60-AV.

Use may also be made of talc particles, for example sold under the names Luzenac Pharma M and UM by Imerys and Rose Talc and Talc SG-2000 by Nippon Talc; natural or synthetic mica particles, such as those sold under the names Mica M RP and Silk Mica by Merck, or the product sold under the name Sericite S-152-BC by Miyoshi Kasei; calcium carbonate and magnesium hydrogen carbonate; hydroxyapatite; boron nitride; fluorphlogopite; and mixtures thereof.

The spherical fillers may be coated with a hydrophobic treatment agent. The hydrophobic treatment agent may be chosen from fatty acids, for instance stearic acid; metal soaps, for instance aluminium dimyristate, the aluminium salt of hydrogenated tallow glutamate; amino acids; N-acylamino acids or salts thereof; lecithin, isopropyl triisostearyl titanate, and mixtures thereof. The N-acylamino acids can comprise an acyl group having from 8 to 22 carbon atoms, such as, for example, a 2-ethylhexanoyl, caproyl, lauroyl, myristoyl, palmitoyl, stearoyl or cocoyl group. The salts of these compounds can be the aluminium, magnesium, calcium, zirconium, zinc, sodium or potassium salts. The amino acid may be, for example, lysine, glutamic acid or alanine. The term “alkyl” mentioned in the compounds cited above denotes in particular an alkyl group having from 1 to 30 carbon atoms and preferably having from 5 to 16 carbon atoms.

If the composition according to the invention contains it/them, the content of filler(s) advantageously represents from 0.5% to 15% by weight and more particularly from 2% to 10% by weight, relative to the weight of the composition.

### Mineral Thickeners

The composition according to the invention can also comprise at least one mineral thickener chosen from optionally modified clays, optionally modified silicas, or mixtures thereof.

More particularly, if the competition contains it, the content of mineral thickener represents from 0.1% to 10% by weight, expressed as active material, and preferably from 0.2% to 5% by weight, relative to the total weight of the composition.

### Optionally Modified Clays

Clays are silicates containing a cation that may be chosen from calcium, magnesium, aluminium, sodium, potassium and lithium cations, and mixtures thereof.

Mention may be made, as examples of such products, of clays of the family of the smectites, and also of the family of the vermiculites, stevensite or chlorites. These clays may be of natural or synthetic origin.

Preferably, use is made of organophilic clays, more particularly of modified clays, such as montmorillonite, bentonite, hectorite, attapulgite or sepiolite, and mixtures thereof. The clay is preferably a bentonite or a hectorite.

These clays are modified with a chemical compound chosen from quaternary amines, tertiary amines, amine acetates, imidazolines, amine soaps, fatty sulfates, alkylarylsulfonates or amine oxides, and mixtures thereof.

Mention may thus be made of hectorites modified by a quaternary amine, more specifically by a C₁₀ to C₂₂ fatty acid ammonium halide, such as chloride, comprising or not comprising an aromatic group, such as hectorite modified by a distearyldimethylammonium halide, preferably chloride (CTFA name: Disteardimonium hectorite), such as, for example, that sold under the name Bentone 38V, Bentone 38V CG or Bentone E W CE by Elementis, or stearalkonium hectorites, such as in particular the product Bentone 27 V.

Mention may also be made of quaternium-18 bentonites, such as those sold, inter alia, under the names Bentone 34 by Elementis, Claytone 40, Tixogel VP by United Catalyst by Southern Clay; stearalkonium bentonites, such as those sold under the names Tixogel LG by United Catalyst and Claytone AF and Claytone APA by Southern Clay; or quaternium-18/benzalkonium bentonites, such as those sold under the name Claytone HT by Southern Clay.

According to one preferred embodiment, the thickening agent is chosen from organophilic modified clays, in particular organophilic modified hectorites, in particular modified by benzyldimethylammonium stearate or distearyldimethylammonium halides, in particular chlorides.

In accordance with an alternative form of the invention, the content of optionally modified clay ranges from 0.2% to 10% by weight, relative to the weight of the composition, and preferably from 0.5% to 5% by weight, relative to the weight of the composition. These percentages are expressed as active material.

### Optionally Modified Silicas

Mention may also be made of fumed silica preferably hydrophobically treated at the surface, the size of the particles of which is advantageously less than 1 μm. It is in fact possible to chemically modify the surface of the silica, by chemical reaction generating a reduction in the number of silanol groups present at the surface of the silica. It is notably possible to substitute silanol groups with hydrophobic groups: a hydrophobic silica is then obtained. The hydrophobic groups may be:
trimethylsiloxy groups, which are notably obtained by treating fumed silica in the presence of hexamethyldisilazane. Silicas thus treated are known as Silica Silylate according to the CTFA (6th edition, 1995). They are sold, for example, under the references Aerosil R812® by Degussa, and Cab-O-Sil TS-530® by Cabot,
dimethylsilyloxy or polydimethylsiloxane groups, which are notably obtained by treating fumed silica in the presence of polydimethylsiloxane or dimethyldichlorosilane. Silicas thus treated are known as Silica Dimethyl Silylate according to the CTFA (6th edition, 1995). They are sold, for example, under the references Aerosil R972® and Aerosil R974® by Degussa, and Cab-O-Sil TS-610® and Cab-O-Sil TS-720® by Cabot.

The hydrophobic fumed silica in particular has a particle size that may be nanometric to micrometric, for example ranging from about from 5 to 200 nm.

The composition according to the invention can also comprise at least silica aerogel particles.

Silica aerogels are porous materials obtained by replacing (by drying) the liquid component of a silica gel with air.

They are generally synthesized via a sol-gel process in a liquid medium and then dried, usually by extraction with a supercritical fluid, the one most commonly used being supercritical CO₂. This type of drying makes it possible to avoid shrinkage of the pores and of the material. The sol-gel process and the various drying operations are described in detail in Brinker C. J. and Scherer G. W., Sol-Gel Science, New York, Academic Press, 1990.

The hydrophobic silica aerogel particles suitable for the implementation of the invention exhibit a specific surface area per unit of mass (SM) ranging from 500 to 1500 m²/g, preferably from 600 to 1200 m²/g and better still from 600 to 800 m²/g, and a size, expressed as the volume-average diameter (D[0.5]), ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

According to one advantageous embodiment, the hydrophobic silica aerogel particles used in the present invention have a specific surface area per unit of mass (SM) ranging from 600 to 800 m²/g and a size expressed as the volume mean diameter (D[0.5]) ranging from 5 to 20 μm and even better still from 5 to 15 μm.

The specific surface per unit of mass can be determined by the nitrogen absorption method, known as the BET (Brunauer-Emmett-Teller) method, described in The Journal of the American Chemical Society, Vol. 60, page 309, February 1938 and corresponding to the international standard ISO 5794/1 (appendix D). The BET specific surface area corresponds to the total specific surface area of the particles under consideration.

The sizes of the silica aerogel particles may be measured by static light scattering using a commercial particle size analyser such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is in particular described in the publication by Van de Hulst, H. C., Light Scattering by Small Particles, Chapters 9 and 10, Wiley, New York, 1957.

According to one preferred embodiment, the hydrophobic silica aerogel particles used in the present invention have a specific surface area per unit of volume S_{V} ranging from 5 to 60 m²/cm³, preferably from 10 to 50 m²/cm³ and better still from 15 to 40 m²/cm³.

The aerogels which can be used according to the present invention are aerogels of hydrophobic silica, preferably of silylated silica (INCI name: Silica Silylate).

As regards the preparation of hydrophobic silica aerogel particles which have been surface-modified by silylation, reference may be made to document U.S. Pat. No. 7,470,725.

Use will preferably be made of hydrophobic silica aerogel particles surface-modified with trimethylsilyl groups.

As hydrophobic silica aerogels that may be used in the invention, examples that may be mentioned include the aerogel sold under the name VM-2260 (INCI name: Silica silylate), by Dow Corning, the particles of which have an average size of about 1000 microns and a specific surface area per unit of mass ranging from 600 to 800 m²/g.

Mention may also be made of the aerogels sold by Cabot under the references Aerogel TLD 201, Aerogel OGD 201, Aerogel TLD 203, Enova® Aerogel MT 1100 and Enova Aerogel MT 1200.

Use will preferably be made of the aerogel sold under the name VM-2270 (INCI name: Silica silylate) by Dow Corning, the particles of which have an average size ranging from 5-15 microns and a specific surface area per unit of mass ranging from 600 to 800 m²/g.

Preferably, when the composition comprises at least one thickening agent chosen from optionally modified silicas, the latter are chosen from hydrophobic silica aerogel particles.

In accordance with an alternative form of the invention, the content of optionally modified silica ranges from 0.5% to 15% by weight and preferably from 1% to 7% by weight, relative to the weight of the composition. These values are expressed as weight of active material.

Preferably, the mineral thickeners are chosen from organophilic clays, in particular modified hectorites; hydrophobic treated fumed silica; hydrophobic silica aerogels, or mixtures thereof, and more specifically still at least one organophilic modified clay or at least one hydrophobic modified silica, in particular hydrophobic silica aerogels.

### Optional Additives

The composition can comprise at least one optional additive chosen, for example, from film-forming agents; antioxidants; preservatives; fragrances; flavourings; neutralizing agents; emollients; organic thickeners; such as, for example, linear or branched dextrin esters; coalescence agents; moisturizing agents; vitamins; and mixtures thereof.

Of course, a person skilled in the art will take care to choose the optional additional additives and/or their amounts such that the advantageous properties of the composition according to the invention are not, or not substantially, detrimentally affected by the envisaged addition.

The examples which follow serve to illustrate the invention without, however, limiting the scope thereof.

### EXAMPLES

### Example 1

The following compositions, the ingredients of which and their respective contents are collated in the table below, are prepared (the contents are expressed as % by weight of starting material, unless otherwise indicated):

TABLE 1

| | Composition |
|---|---|
| Ingredients (INCI name or chemical name) | 1 |
| C12-15 Alkyl Benzoate (C12/C15 Benzoate (DUB B1215); | 15.0 |
| Stéarinerie Dubois) | |
| VP/Eicosene Copolymer (Antaron V 220F; Ashland) | 5.0 |
| PEG-45/Dodecyl Glycol Copolymer (Elfacos ST 9; Akzo Nobel) | 2.0 |
| Polyethylene (Performalene 500-L Polyethylene; NuCera Solutions) | 3.0 |
| Isohexadecane | 10.0 |
| Trimethylsiloxyphenyl Dimethicone (Belsil PDM 1000; Wacker) | 30.0 |
| Mixture of Paraffin, Microcrystalline Wax, Synthetic Wax (Paracera | 8.0 |
| 30540; Paramelt) | |
| Red 7 Lake (and) Isopropyl Titanium Triisostearate (and) | 2.2 |
| Triethoxysilylethyl Polydimethylsiloxyethyl Dimethicone (RED | |
| 7CA C-TTB2; Kobo) | |
| Yellow 6 Lake (and) Isopropyl Titanium Triisostearate (and) | 8.0 |
| Triethoxysilylethyl Polydimethylsiloxyethyl Dimethicone (YELLOW | |
| 6AL C-TTB2; Kobo) | |
| Ethylcellulose (Aqualon EC N7 Pharm; Ashland) | 4.0 |
| Octyldodecanol | qs 100% |
| Ratio | 0.9 |

### Preparation of the Compositions

The ethylcellulose is dispersed in a portion of the octyldodecanol and of the C₁₂-C₁₅ alkyl benzoate, at 105 °C, with Rayneri stirring (speed of the stirring sufficient to have a vortex).

Once the ethylcellulose has been solubilized, the remainder of the octyldodecanol and of the C₁₂-C₁₅ alkyl benzoate, the VP/Eicosene Copolymer, the PEG-45/Dodecyl Glycol Copolymer and the waxes, under the same temperature and stirring conditions.

After homogenization, the phenylated silicone is added, at 105 °C, with Rayneri stirring for at least 15 minutes.

Finally, the isohexadecane is added with stirring at 105 °C

Once the resulting mixture has been homogenized, the pigments are added with stirring.

The resulting mixture is poured under hot conditions into the lipstick mould preheated to 42 °C and the assembly is cooled down to 4 °C Finally, the composition is removed from the mould and the sticks are conditioned.

### Evaluation of the Compositions:

### Stability

The stability of the compositions is evaluated by storing the composition at ambient temperature for 72 hours and by observing if there is exudation of one or more oils.

### Hardness:

The hardness is measured according to the protocol defined above in the description.

### Transfer Resistance:

The lips are made up by performing two passes.

After waiting 1 hour without eating or drinking anything, the lips are pressed on a sheet of white photocopier paper with a grammage of 80 g/m² (kiss test).

The colour deposited on the paper is evaluated visually.

The colour deposited on the paper could also be measured by means of a CIE Lab colorimeter.

### Measurement of the Gloss:

The gloss is measured by evaluating the reflections of the deposit on a support.

The measurement of the gloss is carried out using a BYK Gardner Micro-Tri-Gloss glossmeter and at 3 angles: 20°, 60° and 85°. The index of light reflected on the product is calculated in gloss units (GU).

To do that, the formulation is spread on an Erichsen contrast card in an amount sufficient to be able to obtain a film thickness of 100 μm using a spreader. The measurement of the gloss is taken on the white part of the card 1 hour after spreading.

### Measurement of the Tack:

A sufficient amount of composition is sampled and then squashed in a stainless steel container with a depth of 100 microns.

The composition is then levelled off before taking the measurement.

The containers are left to dry at ambient temperature for 1 hour.

The apparatus used is a TAXT2i texture analyser. The clamp mounted on the apparatus grips an AU4G cylinder with a diameter of 6 mm, at the end of which is adhesively bonded a tip made of smooth beige material which imitates the skin, with the same diameter and a thickness of 2 mm.

Between each measurement, the tip is cleaned with ethanol. The measurements are never taken at the same place in the sample.

The parameters of the compression tests with the maintenance times are given below:

TABLE 2

| Approach speed (or pre-speed) | 1 | mm/s |
|---|---|---|
| Speed (from the detection of contact) | 0.1 | mm/s |
| Force (and corresponding pressure) | 0.283N (namely 0.01 MPa) | |
| Contact time | 3 | seconds |
| Withdrawal speed (or post-speed) | 0.1 | mm/s |

### Results:

The composition obtained is stable.

It is easy to apply, with good glide characteristics, and does not drag, making it possible to have a precise, fine, intense, covering, non-tacky and non-migrating deposit.

The deposit obtained is comfortable and remains so over time, without any feeling of dryness or tautness.

The deposit is glossy with good wear of this glossiness, and with good transfer resistance.

### Example 2

The following compositions, the ingredients of which and their respective contents are collated in the table below, are prepared (the contents are expressed as % by weight of starting material, unless otherwise indicated):

TABLE 3

| | Composition |
|---|---|
| Ingredients (INCI name or chemical name) | 2 |
| Hydrogenated Polyisobutene (Parleam; Nippon Oil & Fats) | 10.0 |
| C12-15 Alkyl Benzoate (C12/C15 Benzoate (DUB B1215); | 15.0 |
| Stéarinerie Dubois) | |
| VP/Eicosene Copolymer (Antaron V 220F; Ashland) | 5.0 |
| PEG-45/Dodecyl Glycol Copolymer (Elfacos ST 9; Akzo Nobel) | 2.0 |
| Polyethylene (Performalene 500-L Polyethylene; NuCera Solutions) | 2.4 |
| Trimethylsiloxyphenyl Dimethicone (Belsil PDM 1000; Wacker) | 30.0 |
| Methyl Methacrylate Crosspolymer (Covabead LH 85. Sensient) | 1.0 |
| Mixture of Paraffin, Microcrystalline Wax, Synthetic Wax (Paracera | 6.5 |
| 30540; Paramelt) | |
| Red 7 Lake (and) Isopropyl Titanium Triisostearate (and) | 2.2 |
| Triethoxysilylethyl Polydimethylsiloxyethyl Dimethicone (RED | |
| 7CA C-TTB2; Kobo) | |
| Yellow 6 Lake (and) Isopropyl Titanium Triisostearate (and) | 8.0 |
| Triethoxysilylethyl Polydimethylsiloxyethyl Dimethicone (YELLOW | |
| 6AL C-TTB2; Kobo) | |
| Ethylcellulose (Aqualon EC N7 Pharm; Ashland) | 4.0 |
| Octyldodecanol | qs 100% |
| ratio | 0.96 |

### Preparation of the Compositions

The ethylcellulose is dispersed in a portion of the octyldodecanol and of the C₁₂-C₁₅ alkyl benzoate, at 105 °C, with Rayneri stirring (speed of the stirring sufficient to have a vortex).

Once the ethylcellulose has been solubilized, the remainder of the octyldodecanol and of the C₁₂-C₁₅ alkyl benzoate, the VP/Eicosene Copolymer, the PEG-45/Dodecyl Glycol Copolymer and the waxes, under the same temperature and stirring conditions.

After homogenization, the phenylated silicone is added, at 105 °C, with Rayneri stirring for at least 15 minutes.

Finally, the hydrogenated polyisobutene is added with stirring at 105 °C

Once the resulting mixture has been homogenized, the pigments and the methyl methacrylate crosspolymer filler are added with stirring.

The resulting mixture is poured under hot conditions into the lipstick mould preheated to 42 °C and the assembly is cooled down to 4 °C Finally, the composition is removed from the mould and the sticks are conditioned.

### Results:

The composition obtained is stable.

It is easy to apply, with good glide characteristics, and does not drag, making it possible to have a precise, fine, intense, covering, non-tacky and non-migrating deposit.

The deposit obtained is comfortable and remains so over time, without any feeling of dryness or tautness.

The deposit is glossy with good wear of this glossiness, and with good transfer resistance.

### Example 3

The following compositions, the ingredients of which and their respective contents are collated in the table below, are prepared (the contents are expressed as % by weight of starting material, unless otherwise indicated):

TABLE 4

| | Composition |
|---|---|
| Ingredients (INCI name or chemical name) | 3 |
| Hydrogenated Polyisobutene (Parleam; Nippon Oil & Fats) | 10.0 |
| C12-15 Alkyl Benzoate (C12/C15 Benzoate (DUB B1215); Stéarinerie | 15.0 |
| Dubois) | |
| Trihydroxystearin (Thixcin R; Elementis) | 0.5 |
| VP/Eicosene Copolymer (Antaron V 220F; Ashland) | 5.0 |
| PEG-45/Dodecyl Glycol Copolymer (Elfacos ST 9; Akzo Nobel) | 2.0 |
| Polyethylene (Performalene 500-L Polyethylene; NuCera Solutions) | 2.7 |
| Trimethylsiloxyphenyl Dimethicone (Belsil PDM 1000; Wacker) | 30.0 |
| Methyl methacrylate crosspolymer | 1.0 |
| Mixture of Paraffin, Microcrystalline Wax, Synthetic Wax (Paracera | 7.2 |
| 30540; Paramelt | |
| Red 7 Lake (and) Isopropyl Titanium Triisostearate (and) | 6.7 |
| Triethoxysilylethyl Polydimethylsiloxyethyl Dimethicone (RED | |
| 7CA C-TTB2; Kobo) | |
| Yellow 6 Lake (and) Isopropyl Titanium Triisostearate (and) | 1.3 |
| Triethoxysilylethyl Polydimethylsiloxyethyl Dimethicone (YELLOW | |
| 6AL C-TTB2; Kobo) | |
| Titanium Dioxide (and) Alumina (and) Isopropyl Titanium | 3.6 |
| Triisostearate (and) Triethoxysilylethyl Polydimethylsiloxyethyl | |
| Dimethicone (TTO-TTB7; Kobo) | |
| Ethylcellulose (Aqualon EC N7 Pharm; Ashland) | 4.0 |
| Octyldodecanol | qs 100% |
| ratio | 0.86 |

### Preparation of the Compositions

The ethylcellulose is dispersed in a portion of the octyldodecanol and of the C₁₂-C₁₅ alkyl benzoate, at 105 °C, with Rayneri stirring (speed of the stirring sufficient to have a vortex).

Once the ethylcellulose has been solubilized, the remainder of the octyldodecanol and of the C₁₂-C₁₅ alkyl benzoate, the VP/Eicosene Copolymer, the PEG-45/Dodecyl Glycol Copolymer and the waxes, under the same temperature and stirring conditions.

After homogenization, the phenylated silicone is added, at 105 °C, with Rayneri stirring for at least 15 minutes.

Finally, the hydrogenated polyisobutene is added with stirring at 105 °C

Once the resulting mixture has been homogenized, the pigments are added with stirring.

The resulting mixture is poured under hot conditions into the lipstick mould preheated to 42 °C and the assembly is cooled down to 4 °C Finally, the composition is removed from the mould and the sticks are conditioned.

### Results:

The composition obtained is stable.

It is easy to apply, with good glide characteristics, and does not drag, making it possible to have a precise, fine, intense, covering, non-tacky and non-migrating deposit.

The deposit obtained is comfortable and remains so over time, without any feeling of dryness or tautness.

The deposit is glossy with good wear of this glossiness, and with good transfer resistance.

### Example 4

The following compositions, the ingredients of which and their respective contents are collated in the table below, are prepared (the contents are expressed as % by weight of starting material, unless otherwise indicated):

TABLE 5

| | Composition |
|---|---|
| Ingredients (INCI name or chemical name) | 4 |
| C12-15 Alkyl Benzoate (C12/C15 Benzoate (DUB B1215); | 15.0 |
| Stéarinerie Dubois) | |
| VP/Eicosene Copolymer (Antaron V 220F; Ashland) | 5.0 |
| PEG-45/Dodecyl Glycol Copolymer (Elfacos ST 9; Akzo Nobel) | 2.0 |
| Polyethylene (Performalene 500-L Polyethylene; NuCera Solutions) | 2.7 |
| Isohexadecane | 10.0 |
| Trimethylsiloxyphenyl Dimethicone (Belsil PDM 1000; Wacker) | 30.0 |
| Mixture of Paraffin, Microcrystalline Wax, Synthetic Wax (Paracera | 7.2 |
| 30540; Paramelt) | |
| Red 7 Lake (and) Isopropyl Titanium Triisostearate (and) | 6.7 |
| Triethoxysilylethyl Polydimethylsiloxyethyl Dimethicone (RED | |
| 7CA C-TTB2; Kobo) | |
| Yellow 6 Lake (and) Isopropyl Titanium Triisostearate (and) | 1.30 |
| Triethoxysilylethyl Polydimethylsiloxyethyl Dimethicone (YELLOW | |
| 6AL C-TTB2; Kobo) | |
| Titanium Dioxide (and) Alumina (and) Isopropyl Titanium | 3.6 |
| Triisostearate (and) Triethoxysilylethyl Polydimethylsiloxyethyl | |
| Dimethicone (TTO-TTB7; Kobo) | |
| Ethylcellulose (Aqualon EC N7 Pharm; Ashland) | 4.0 |
| Octyldodecanol | qs 100% |
| ratio | 0.92 |

### Preparation of the Compositions

The ethylcellulose is dispersed in a portion of the octyldodecanol and of the C₁₂-C₁₅ alkyl benzoate, at 105 °C, with Rayneri stirring (speed of the stirring sufficient to have a vortex).

Once the ethylcellulose has been solubilized, the remainder of the octyldodecanol and of the C₁₂-C₁₅ alkyl benzoate, the VP/Eicosene Copolymer, the PEG-45/Dodecyl Glycol Copolymer and the waxes, under the same temperature and stirring conditions.

After homogenization, the phenylated silicone is added, at 105 °C, with Rayneri stirring for at least 15 minutes.

Finally, the isohexadecane is added with stirring at 105 °C

Once the resulting mixture has been homogenized, the pigments are added with stirring.

The resulting mixture is poured under hot conditions into the lipstick mould preheated to 42 °C and the assembly is cooled down to 4 °C Finally, the composition is removed from the mould and the sticks are conditioned.

### Results:

The composition obtained is stable.

It is easy to apply, with good glide characteristics, and does not drag, making it possible to have a precise, fine, intense, covering, non-tacky and non-migrating deposit.

The deposit obtained is comfortable and remains so over time, without any feeling of dryness or tautness.

The deposit is glossy with good wear of this glossiness, and with good transfer resistance.

### Example 5

The following compositions, the ingredients of which and their respective contents are collated in the table below, are prepared (the contents are expressed as % by weight of starting material, unless otherwise indicated):

TABLE 6

| | Composition |
|---|---|
| Ingredients (INCI name or chemical name) | 5 |
| C12-15 Alkyl Benzoate (C12/C15 Benzoate (DUB B1215); Stéarinerie | 15.0 |
| Dubois) | |
| VP/Eicosene Copolymer (Antaron V 220F; Ashland) | 5.0 |
| PEG-45/Dodecyl Glycol Copolymer (Elfacos ST 9; Akzo Nobel) | 2.0 |
| Polyethylene (Performalene 500-L Polyethylene; NuCera Solutions) | 3.0 |
| Isohexadecane | 10.0 |
| Trimethylsiloxyphenyl Dimethicone (Belsil PDM 1000; Wacker) | 30.0 |
| Mixture of Paraffin, Microcrystalline Wax, Synthetic Wax (Paracera | 8.0 |
| 30540; Paramelt) | |
| Red 7 Lake (and) Isopropyl Titanium Triisostearate (and) | 2.2 |
| Triethoxysilylethyl Polydimethylsiloxyethyl Dimethicone (RED | |
| 7CA C-TTB2; Kobo) | |
| Yellow 6 Lake (and) Isopropyl Titanium Triisostearate (and) | 8.0 |
| Triethoxysilylethyl Polydimethylsiloxyethyl Dimethicone (YELLOW | |
| 6AL C-TTB2; Kobo) | |
| Ethylcellulose (Aqualon EC N7 Pharm; Ashland) | 4.0 |
| Octyldodecanol | qs 100% |
| ratio | 0.93 |

### Preparation of the Compositions

The ethylcellulose is dispersed in a portion of the octyldodecanol and of the C₁₂-C₁₅ alkyl benzoate, at 105 °C, with Rayneri stirring (speed of the stirring sufficient to have a vortex).

Once the ethylcellulose has been solubilized, the remainder of the octyldodecanol and of the C₁₂-C₁₅ alkyl benzoate, the VP/Eicosene Copolymer, the PEG-45/Dodecyl Glycol Copolymer and the waxes, under the same temperature and stirring conditions.

After homogenization, the phenylated silicone is added, at 105 °C, with Rayneri stirring for at least 15 minutes.

Finally, the isohexadecane is added with stirring at 105 °C

Once the resulting mixture has been homogenized, the pigments are added with stirring.

The resulting mixture is poured under hot conditions into the lipstick mould preheated to 42 °C and the assembly is cooled down to 4 °C Finally, the composition is removed from the mould and the sticks are conditioned.

### Results:

The composition obtained is stable.

It is easy to apply, with good glide characteristics, and does not drag, making it possible to have a precise, fine, intense, covering, non-tacky and non-migrating deposit.

The deposit obtained is comfortable and remains so over time, without any feeling of dryness or tautness.

The deposit is glossy with good wear of this glossiness, and with good transfer resistance.

1. Solid cosmetic composition for making up human keratin materials, in particular the skin and/or the lips, comprising:
at least ethylcellulose,
at least a first oil, which is chosen from esters of benzoic acid and of a monoalcohol, which is preferably saturated, of C8-C20, preferably C8-C18, and also mixtures thereof,
at least a second oil, which is a polar non-volatile hydrocarbon-based oil chosen from C10-C26 alcohols and optionally from esters other than the first oil(s),
at least a third oil, which is chosen from non-volatile phenylated silicones comprising at least one dimethicone fragment,
optionally at least a fourth oil, which is a non-volatile non-phenylated silicone oil or a non-volatile phenylated silicone oil with no dimethicone fragment,
the contents of oils being such that the weight ratio of the first oil(s), second oil(s) and where appropriate fourth oil(s)/third oil(s) is less than or equal to 1.

2. Composition according to the preceding claim, characterized in that the ethylcellulose content is between 1% and 10% by weight, relative to the weight of the composition.

3. Composition according to claim 1, characterized in that the first oil is chosen from benzoic acid monoesters chosen from C₁₂-C₁₅ alkyl benzoate, C₁₆-C₁₇ alkyl benzoate, stearyl benzoate, isostearyl benzoate, ethylhexyl benzoate, octyldodecyl benzoate, and mixtures thereof.

4. Composition according to claim 1, characterized in that the content of first oil(s) is between 8% and 30% by weight relative to the total weight of the composition.

5. Composition according to claim 1, characterized in that the non-volatile hydrocarbon-based second oil is chosen from monohydroxylated C10-C26 alcohols.

6. Composition according to claim 1, characterized in that the non-volatile hydrocarbon-based second oil can also comprise at least one ester other than the first oil(s), chosen from aromatic, saturated or unsaturated, linear or branched mono- or diesters comprising more particularly from 12 to 80 carbon atoms, and optionally one or two ether groups; aromatic, saturated or unsaturated, linear or branched triesters, comprising up to 80 carbon atoms, optionally comprising one to three ether groups;
tetraesters comprising from 35 to 80 carbon atoms, optionally comprising one to three ether groups; polyesters obtained by condensation of an unsaturated fatty acid dimer and/or trimer and of a diol; esters of a dimer diol or of a polyol and of a mono- and/or dicarboxylic acid; vegetable oils, and mixtures thereof.

7. Composition according to claim 1, characterized in that the content of second oil(s) ranges from 8% to 20% by weight relative to the total weight of the composition.

8. Composition according to claim 1, characterized in that the content of C10-C26 alcohols ranges from 8% to 20% by weight, relative to the total weight of the composition.

9. Composition according to claim 1, characterized in that the (ethylcellulose/(ethylcellulose+C10-C26 alcohol(s)) weight ratio*100 is at least 10%.

10. Composition according to claim 1, characterized in that the non-volatile phenylated silicone third oil(s) comprising at least one dimethicone group is/are chosen from Trimethylsiloxyphenyl Dimethicones, Diphenyl Dimethicones, Tetramethyl Tetraphenyl Trisiloxanes and mixtures thereof.

11. Composition according to claim 1, characterized in that the content of non-volatile phenylated silicone third oil(s) represents from 20% to 40% by weight relative to the total weight of the composition.

12. Composition according to claim 1, characterized in that it comprises at least a fourth oil, which is a non-volatile phenylated silicone oil with no dimethicone fragment or a non-volatile non-phenylated silicone oil.

13. Composition according to claim 1, characterized in that the content of fourth oil(s), if the composition comprises same present, is less than or equal to 5% by weight relative to the total weight of the composition.

14. Composition according to claim 1, characterized in that the content of oils is such that the weight ratio of the first oil(s), second oil(s) and where appropriate fourth oil(s)/third oil(s) is between 0.5 and 1.

15. Composition according to claim 1, characterized in that it comprises at least one non-volatile, non-polar hydrocarbon-based oil.

16. Composition according to the preceding claim, characterized in that the content of non-volatile, non-polar hydrocarbon-based oil(s) represents from 0.1% to 15% by weight relative to the total weight of the composition.

17. Composition according to claim 1, characterized in that it comprises at least one volatile hydrocarbon-based or silicone oil or combinations thereof.

18. Composition according to the preceding claim, characterized in that the content of volatile oil(s) represents from 0.1% to 15% by weight relative to the total weight of the composition.

19. Composition according to claim 1, characterized in that the composition comprises at least one wax.

20. Composition according to the preceding claim, characterized in that the wax is chosen from non-polar waxes; polar hydrocarbon-based waxes; alcohol waxes; and mixtures thereof.

21. Composition according to the preceding claim, characterized in that the content of wax ranges from 1% to 20% by weight, relative to the total weight of the composition.

22. Composition according to claim 1, characterized in that it comprises at least one compound which is pasty at ambient temperature and atmospheric pressure.

23. Composition according to the preceding claim, characterized in that the content of pasty compound ranges from 0.5% to 15% by weight relative to the total weight of the composition.

24. Composition according to claim 1, characterized in that it comprises less than 2% by weight of water, relative to the weight of the composition.

25. Composition according to claim 1, characterized in that it comprises at least one colorant.

26. Composition according to claim 1, characterized in that it comprises at least one mineral thickener chosen from optionally modified clays, optionally modified silicas, or mixtures thereof.

27. Process for making up and/or caring for the lips, comprising applying the composition according to claim 1 to the lips.
